(19) United States
(12) Patent Application Publication
(10) Pub. No.: US 2005/0136500 A1
(43) Pub. Date: Jun. 23, 2005

(54) Flow-through assay devices

(75) Inventors: Kaiyuan Yang, Cumming, GA (US); RameshBabu Boga, Roswell, GA (US); Shawn Ray Feaster, Duluth, GA (US); Rosann Marie Matthews Kaylor, Cumming, GA (US); David Samuel Cohen, Alpharetta, GA (US)

Correspondence Address: DORITY & MANNING, P.A., POST OFFICE BOX 1449, GREENVILLE, SC, 29602-1449, US

(73) Assignee: Kimberly-Clark Worldwide; Inc.

(21) Appl. No.: 10/742,590

(22) Filed: Dec. 19, 2003

(51) Int. Cl. C12Q 1/54; G01N 33/558; G01N 33/543

(52) U.S. Cl. 435/14; 436/514

(57) ABSTRACT

A flow-through assay device capable of detecting the presence or quantity of an analyte of interest is provided that is accurate, reliable, and easy-to-use. The device contains a substrate printed with a channel to facilitate the flow of a test sample to a detection working electrode. The detection working electrode communicates with affinity reagents, such as redox mediators and capture ligands. For instance, capture ligands that are specific binding members for the analyte of interest are applied to the detection electrode to serve as the primary location for detection of the analyte.

### BACKGROUND OF THE INVENTION

Various analytical procedures and devices are commonly employed in assays to determine the presence and/or absence of analytes in a test sample. For instance, immunoassays utilize mechanisms of the immune systems, wherein antibodies are produced in response to the presence of antigens that are pathogenic or foreign to the organisms. These antibodies and antigens, i.e., immunoreactants, are capable of binding with one another, thereby causing a highly specific reaction mechanism that may be used to determine the presence or concentration of that particular antigen in a biological sample. There are several well-known techniques for detecting the presence of an analyte.

One such technique is described in WO 01/38873 to Zhang, Zhanq describes flow-through electrochemical biosensors designed to detect the presence of an analyte. FIG. 2 of Zhang, for instance, illustrates a sensor assembly 5 that includes an absorbent pad 18, a wicking mesh 22, and a conjugate pad 20 that overlay an application area 14′ and a detection area 16′. The wicking mesh 22 functions as a carrier to deliver the fluid sample through capillary action to the detection area 16′ where the analyte will become immobilized on the electrode surface. In Example 4 of Zhang, various materials of different pore sizes (ranging from 0.63 to 100 microns) were tested to determine the time for a buffer solution to flow 4 centimeters along the membrane. The times ranged from 40 seconds to 3 minutes, 45 seconds. Zhang indicates that any of the membranes tested could be used to provide a rapid test.

Unfortunately, conventional flow-through electrochemical biosensors, such as described above, possess various problems. For instance, traditional flow-through assay devices require a large sample volume because of the presence of a large sampling pad, wicking pad, and porous membrane. Moreover, the contact of the sample with the working electrode surface is not always sufficient because a large portion of the sample does not flow through the electrode surface, rather it flows through the membrane itself and bypasses the working electrode. In addition, other than the membrane, there are no flow control mechanisms. Consequently, if a slow moving membrane (e.g., nitrocellulose) is used, the sample will flow mostly within the membrane and thus leave significant amount of residues (e.g. redox labels) inside the membrane that lead to a large background current. On the other hand, if a fast moving membrane (e.g., nylon mesh) is used, the flow speed may be too fast to handle the data acquisition or necessary reaction time. As such, a need still exits for improved flow-through electrochemical sensors.

### SUMMARY OF THE INVENTION

In accordance with one embodiment of the present invention, a method is disclosed for detecting the presence or quantity of an analyte residing in a test sample. The method comprises:

i) forming a flow-through assay device by a method comprising:
a) printing a fluidic channel onto a substrate;
b) forming a detection working electrode on the substrate, the detection working electrode being in fluid communication with the fluidic channel; and
c) treating a surface of the detection working electrode with a specific binding capture ligand for the analyte; and

ii) contacting the test sample with the fluidic channel of the assay device.

In accordance with another embodiment of the present invention, a method is disclosed for detecting the presence or quantity of an analyte residing in a test sample, the method comprising:

i) forming a flow-through assay device by a method comprising:
a) printing a fluidic microchannel onto a substrate, the fluidic microchannel being formed from a dielectric material and having a characteristic dimension of from about 0.5 to about 500 micrometers;
b) forming a detection working electrode on the substrate, the detection working electrode being in fluid communication with the fluidic microchannel; and
c) treating a surface of the detection working electrode with a specific binding capture ligand for the analyte;

ii) contacting the test sample with the fluidic microchannel of the assay device;

iii) applying a potential difference between the detection working electrode and a counter electrode; and

iv) measuring the current generated at the detection working electrode.

In accordance with still another embodiment of the present invention, a flow-through assay device is disclosed for detecting the presence or quantity of an analyte residing in a test sample. The flow-through assay device comprises a fluidic microchannel printed onto a substrate that facilitates the flow of the test sample to a detection working electrode. The microchannel has a characteristic dimension of from about 0.5 to about 500 micrometers. A specific binding capture ligand for the analyte is applied to the detection working electrode. The detection working electrode is capable of generating a measurable detection current, wherein the amount of the analyte within the test sample is proportional to the detection current generated at the detection working electrode.

Other features and aspects of the present invention are discussed in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 is a schematic illustration of one embodiment of a flow-through assay device of the present invention;

FIG. 2 is a schematic illustration of another embodiment of a flow-through assay device of the present invention; and

FIG. 3 illustrates the “periodate” method of forming a horseradish peroxidase (HRP) conjugate for use in one embodiment of the present invention.

Repeat use of reference characters in the present specification and drawings is intended to represent same or analogous features or elements of the invention.

### DETAILED DESCRIPTION OF REPRESENTATIVE EMBODIMENTS

### Definitions

As used herein, the term “analyte” generally refers to a substance to be detected. For instance, analytes may include antigenic substances, haptens, antibodies, and combinations thereof. Analytes include, but are not limited to, toxins, organic compounds, proteins, peptides, microorganisms, amino acids, nucleic acids, hormones, steroids, vitamins, drugs (including those administered for therapeutic purposes as well as those administered for illicit purposes), drug intermediaries or byproducts, bacteria, virus particles and metabolites of or antibodies to any of the above substances. Specific examples of some analytes include ferritin; creatinine kinase MB (CK-MB); digoxin; phenytoin; phenobarbitol; carbamazepine; vancomycin; gentamycin; theophylline; valproic acid; quinidine; luteinizing hormone (LH); follicle stimulating hormone (FSH); estradiol, progesterone; C-reactive protein; lipocalins; IgE antibodies; cytokines; vitamin B2 micro-globulin; glycated hemoglobin (Gly. Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-IgG) and toxoplasmosis IgM (Toxo-IgM); testosterone; salicylates; acetaminophen; hepatitis B virus surface antigen (HBsAg); antibodies to hepatitis B core antigen, such as anti-hepatitis B core antigen IgG and IgM (Anti-HBC); human immune deficiency virus 1 and 2 (HIV 1 and 2); human T-cell leukemia virus 1 and 2 (HTLV); hepatitis B e antigen (HBeAg); antibodies to hepatitis B e antigen (Anti-HBe); influenza virus; thyroid stimulating hormone (TSH); thyroxine (T4); total triiodothyronine (Total T3); free triiodothyronine (Free T3); carcinoembryoic antigen (CEA); lipoproteins, cholesterol, and triglycerides; and alpha fetoprotein (AFP). Drugs of abuse and controlled substances include, but are not intended to be limited to, amphetamine; methamphetamine; barbiturates, such as amobarbital, secobarbital, pentobarbital, phenobarbital, and barbital; benzodiazepines, such as librium and valium; cannabinoids, such as hashish and marijuana; cocaine; fentanyl; LSD; methaqualone; opiates, such as heroin, morphine, codeine, hydromorphone, hydrocodone, methadone, oxycodone, oxymorphone and opium; phencyclidine; and propoxyhene. Other potential analytes may be described in U.S. Pat. No. 6,436,651 to Everhart, et al. and U.S. Pat. No. 4,366,241 to Tom et al.

As used herein, the term “test sample” generally refers to a material suspected of containing the analyte. The test sample may be used directly as obtained from the source or following a pretreatment to modify the character of the sample. The test sample may be derived from any biological source, such as a physiological fluid, including, blood, interstitial fluid, saliva, ocular lens fluid, cerebral spinal fluid, sweat, urine, milk, ascites fluid, mucous, synovial fluid, peritoneal fluid, vaginal fluid, amniotic fluid or the like. The test sample may be pretreated prior to use, such as preparing plasma from blood, diluting viscous fluids, and the like. Methods of treatment may involve filtration, precipitation, dilution, distillation, mixing, concentration, inactivation of interfering components, and the addition of reagents. Besides physiological fluids, other liquid samples may be used such as water, food products and the like for the performance of environmental or food production assays. In addition, a solid material suspected of containing the analyte may be used as the test sample. In some instances it may be beneficial to modify a solid test sample to form a liquid medium or to release the analyte.

### Detailed Description

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention covers such modifications and variations as come within the scope of the appended claims and their equivalents.

In general, the present invention is directed to a flow-through assay device capable of detecting the presence or quantity of an analyte of interest that is accurate, reliable, and easy-to-use. The device contains a substrate printed with a channel to facilitate the flow of a test sample to a detection working electrode. The detection working electrode communicates with affinity reagents, such as redox mediators and capture ligands. For instance, capture ligands that are specific binding members for the analyte of interest are applied to the detection electrode to serve as the primary location for detection of the analyte.

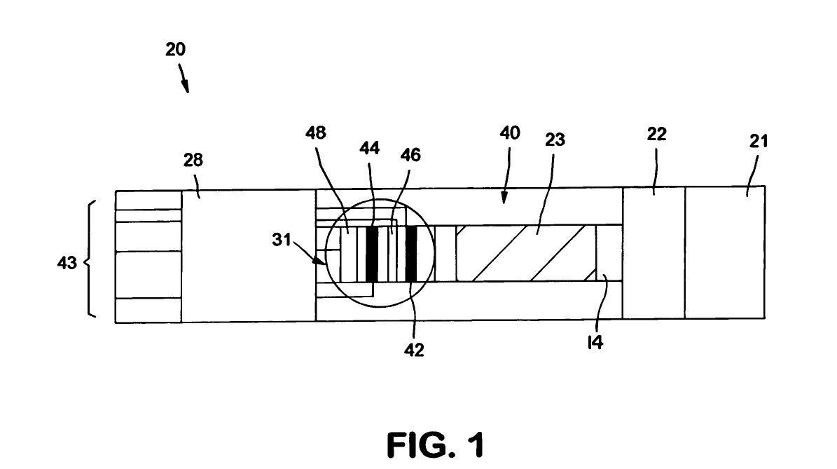

Referring to FIG. 1, for instance, one embodiment of a flow-through assay device 20 that may be formed according to the present invention will now be described in more detail. As shown, the device 20 contains a substrate 40 that may be formed from an insulative material, such as silicon, fused silicon dioxide, silicate glass, alumina, aluminosilicate ceramic, an epoxy, an epoxy composite such as glass fiber reinforced epoxy, polyester, polyimide, polyamide, polycarbonate, etc. In this embodiment, a sample pad 21 is provided to receive the test sample. Alternatively, other devices, such as reaction chambers or wells, may be provided to receive the test sample. Some suitable materials that may be used to form the sample pad 21 include, but are not limited to, nitrocellulose, cellulose, porous polyethylene or polypropylene pads, and glass fibers. If desired, the sample pad 21 may contain one or more assay pretreatment reagents, either covalently or non-covalently attached thereto. The device 20 may also contain a wicking pad 28. The wicking pad 28 generally receives fluid that has migrated through the device 20. As is well known in the art, the wicking pad 28 may assist in promoting capillary action and fluid flow.

Regardless of the manner in which the assay device 20 is generally constructed, a sample channel 14 is formed on the substrate 40 that acts as a fluidic medium to transport the test sample to a detection zone 31. Multiple sample channels 14 may be utilized for multiple test samples. The sample channel 14 may be formed from any of a variety of materials through which the test sample is capable of flowing. In most embodiments, it is desired that a dielectric material be used to form the sample channel 14 to reduce unwanted interference with the electrochemical detection of the analyte. The term “dielectric” material generally refers to a material having a dielectric constant “k” of less than about 5 at 1 kHz (defined by ASTM D150-98 Standard Test Methods for AC Loss Characteristics and Permittivity (Dielectric Constant) of Solid Electrical Insulation, an insulation resistance of greater than 10 GΩ/mil, and/or a breakdown voltage of greater than 1000 V/mil DC (also defined by ASTM D150-98 Standard Test Methods for AC Loss Characteristics and Permittivity (Dielectric Constant) of Solid Electrical Insulation. For example, a wide variety of organic and inorganic polymers, both natural and synthetic may be employed as a dielectric material for the sample channel 14. Examples of such polymers include, but are not limited to, polyesters, polyimides, polyamides, polycarbonates, polyolefins (e.g., polyethylene, polypropylene, etc.), polysiloxanes, polyurethanes, polyvinylchlorides, polystyrenes, and so forth. Commercial dielectric materials, such as 5036 Heat Seal/Encapsulant, 5018 UV curable dielectric, 5018G UV curable dielectric and 5018A UV curable dielectric are available from DuPont Biosensor Group of Research Triangle Park, North Carolina.

If desired, such a polymeric channel may be formed by first applying monomer(s) or pre-polymer(s) for the polymer to the substrate 40, and then polymerizing the monomer(s) or pre-polymer(s) using well-known techniques, such as heating, irradiating, etc. For example, polymerization may be induced with ionizing radiation, which is radiation having an energy sufficient to either directly or indirectly produce ions in a medium. Some suitable examples of ionizing radiation that may be used in the present invention include, but are not limited to, ultraviolet radiation, electron beam radiation, natural and artificial radio isotopes (e.g., α, β, and γ rays), x-rays, neutron beams, positively charged beams, laser beams, and so forth. Electron beam radiation, for instance, involves the production of accelerated electrons by an electron beam device. Electron beam devices are generally well known in the art. For instance, examples of suitable electron beam devices are described in U.S. Pat. No. 5,003,178 to Livesay; U.S. Pat. No. 5,962,995 to Avnery; U.S. Pat. No. 6,407,492 to Avnery. et al., which are incorporated herein in their entirety by reference thereto for all purposes.

The geometry of the sample channel 14 may be selected so that capillary forces assist the flow of the test sample through the sample channel 14. For example, the sample channel 14 may have a cross-sectional shape that is circular, square, rectangular, triangular, v-shaped, u-shaped, hexagonal, octagonal, irregular, and so forth. The sample channel 14 may also be continuous or discontinuous, and may also contain continuous or discontinuous sample mixing islands to promote sample mixing. Further, in some embodiments, the sample channel 14 may be a “microchannel”, which is a channel that allows for fluid flow in the low Reynolds number region where fluid dynamics are dominated by viscous forces rather than inertial forces. The formula for Reynolds number is as follows:
Re=ρδ²/ητ+ρμδ/η
wherein, μ is the velocity vector, ρ is the fluid density, η is the viscosity of the fluid, δ is the characteristic dimension of the channel (e.g., diameter, width, etc.), and τ is the time scale over which the velocity changes (where μ/τ=δμ/dt). Fluid flow behavior at steady state (τ→∝) is characterized by the Reynolds number, Re=ρμδ/η. Due to their small size and slow velocity, microchannels often allow fluids to flow in the low Reynolds number regime (Re less than about 1). In this regime, inertial effects, which cause turbulence and secondary flows, are negligible, and viscous effects dominate the dynamics so that flow is generally laminar. Thus, to maintain laminar flow, it is sometimes desired that the characteristic dimension of the channel range from about 0.5 micrometers and about 500 micrometers, in some embodiments from about 1 micrometer to about 200 micrometers, and in some embodiments, from about 5 micrometers to about 100 micrometers.

The height or depth of the sample channel 14 may also vary to accommodate different volumes of the test sample. The sample channel 14 may contain opposing walls that are raised a certain height above the surface of the substrate 40. For example, the walls may have a height of from about 0.1 to about 500 micrometers, in some embodiments from about 0.5 to about 250 micrometers, and in some embodiments, from about 1 to about 100 micrometers. In some embodiments, the height of the sample channel 14 is the combination of the printed channel and an adhesive layer (e.g., glue, double-sided tape, etc.) used, for instance, to laminate a porous membrane over the printed channel. The thickness of the adhesive layer may vary, for instance, from about 10 to about 100 microns. Likewise, the length of the sample channel 14 may also vary. For example, the sample channel 14 may have a length that is from about 1 millimeter to about 50 centimeters, and in some embodiments, from about 5 millimeters to about 50 millimeters.

Printing techniques are generally utilized in the present invention to apply the sample channel 14 to the substrate 40 due to their practical and cost-saving benefits. For instance, several suitable printing techniques are described in U.S. Pat. No. 5,512,131 to Kumar, et al.; U.S. Pat. No. 5,922,550 to Everhart, et al.; U.S. Pat. No. 6,294,392 to Kuhr, et al.; U.S. Pat. No. 6,509,085 to Kennedy; and U.S. Pat. No. 6,573,040 to Everhart, et al., which are incorporated herein in their entirety by reference thereto for all purposes. For example, in one embodiment, “stamp printing” is utilized to apply the sample channel 14 to the substrate 40. Some suitable stamp printing techniques are described in U.S. Pat. No. 5,512,131 to Kumar, et al. and U.S. Pat. No. 5,922,550 to Everhart, et al. For example, an elastomeric stamp may be used to transfer the ink to the substrate surface through contact. The stamp is fabricated by casting polydimethylsiloxane (PDMS) on a master having the inverse of the desired print pattern, which will thereby result in the desired channel pattern. Masters are prepared using standard photolithographic techniques, or constructed from existing materials having microscale surface features. In one embodiment, a photolithographically-produced master is placed in a glass or plastic Petri dish, and a mixture of SYLGARD® silicone elastomer 184 and SYLGARD® silicone elastomer 184 curing agent (Dow Corning Corporation) is poured over it. The polydimethylsiloxane (PDMS) elastomer is allowed to sit at room temperature and is then cured; alternately, for faster curing, the elastomer may be cured at a temperature of from about 60 to about 65° C. When cured, PDMS is sufficiently elastomeric to allow good conformal contact of the stamp and the surface of the substrate 40.

The resulting elastomeric stamp is “inked” by exposing the stamp to a solution of the desired material used to help form the fluidic channel. This is typically done by placing the stamp face down in the solution for about 10 seconds to about 10 minutes. The stamp is allowed to dry, either under ambient conditions or by exposure to a stream of air or nitrogen gas. Following inking, the stamp is applied to the surface of the substrate 40. Light pressure is used to ensure complete contact between the stamp and the substrate 40. After about 1 second to about 5 minutes, the stamp is then gently peeled from the substrate 40. Following removal of the stamp, the substrate 40 may be rinsed and dried.

Stamp printing, such as described above, may be used to prepare channels in various ways. In one embodiment, for example, the elastomeric stamp is inked with a material that significantly alters the surface energy of the substrate 40 so that it may be selectively “wettable” to the monomer or pre-polymer (if post-cured), or polymer used to make the channel. The stamp could have raised features to print the desired channel pattern. An exemplary stamp printing method may involve inking the stamp with a wetting agent, such as hydrophilic self-assembling monolayers (SAMs), including those that are carboxy-terminated. Various examples of such self-assembling monolayers are described in U.S. Pat. No. 5,922,550 to Everhart, et al. In another embodiment, hydrophobic wetting agents may be utilized. Specifically, the inverse of the desired pattern is stamp printed onto a hydrophilic substrate. Upon exposure of the monomer or pre-polymer (if post-cured), or polymer, the inks would selectively wet only on the substrate 40, thereby resulting in the desired channel pattern. Another stamp printing technique might simply involve inking an elastomeric stamp with a solution of the monomer or pre-polymer (if post-cured), or polymer. The stamp may have raised features to match the desired channel pattern so that a direct transfer of the channel-forming material would occur on the substrate 40.

Still another suitable contact printing technique that may be utilized in the present invention is “screen printing.” Screen printing is performed manually or photomechanically. The screens may include a silk or nylon fabric mesh with, for instance, from about 40 to about 120 openings per lineal centimeter. The screen material is attached to a frame and stretched to provide a smooth surface. The stencil is applied to the bottom side of the screen, i.e., the side in contact with the substrate upon which the fluidic channels are to be printed. The print material is painted onto the screen, and transferred by rubbing the screen (which is in contact with the substrate) with a squeegee.

In addition to contact printing, any of a variety of well-known non-contact printing techniques may also be employed in the present invention. In one embodiment, for example, ink-jet printing may be employed. Ink-jet printing is a non-contact printing technique that involves forcing ink through a tiny nozzle (or a series of nozzles) to form droplets that are directed toward the substrate. Two techniques are generally utilized, i.e., “DOD” (Drop-On-Demand) or “continuous” ink-jet printing. In continuous systems, ink is emitted in a continuous stream under pressure through at least one orifice or nozzle. The stream is perturbed by a pressurization actuator to break the stream into droplets at a fixed distance from the orifice. DOD systems, on the other hand, use a pressurization actuator at each orifice to break the ink into droplets. The pressurization actuator in each system may be a piezoelectric crystal, an acoustic device, a thermal device, etc. The selection of the type of ink jet system varies on the type of material to be printed from the print head. For example, conductive materials are sometimes required for continuous systems because the droplets are deflected electrostatically. Thus, when the sample channel is formed from a dielectric material, DOD printing techniques may be more desirable.

In addition to the printing techniques mentioned above, any other suitable printing technique may be used in the present invention. For example, other suitable printing techniques may include, but not limited to, such as laser printing, thermal ribbon printing, piston printing, spray printing, flexographic printing, gravure printing, etc. Such techniques are well known to those skilled in the art.

The sample channel 14 is sometimes formed from a material that is relatively hydrophobic and resistant to the flow of a test sample. To counteract this effect, a porous membrane 23 may be placed in fluid communication with the sample channel 14 to facilitate the flow of the test sample. For example, as shown in FIG. 1, the porous membrane 23 is positioned above the channel 14. Of course, the porous membrane 23 may also be positioned below the channel 14, or in any other position relative to the channel 14.

The pores of the membrane 23 help guide the test sample to a detection working electrode 42 (described below) and may also help facilitate uniform mixing. In some cases, it may be desired that the “flow time” of the test sample through the membrane be long enough to promote uniform mixing and ensure that the analyte within the test sample has sufficient time to react with the desired reagents. For example, the time for the test sample to contact the detection working electrode 42 upon application may be at least about 1 minute, in some embodiments at least about 2 minutes, in some embodiments from about 3 to about 10 minutes, and in some embodiments, from about 4 to about 8 minutes. Such enhanced flow times are not only possible for test samples with high volumes, but also for test samples with low volumes. For example, test samples having a volume of less than about 100 microliters, in some embodiments from about 0.55 to about 50 microliters, and in some embodiments, from about 5 to about 35 microliters, may have an enhanced flow time. The ability to use such small test sample volumes is beneficial in that larger test volumes often increase background interference.

Without intending to be limited by theory, it is believed that the ability to achieve a long flow time for test samples with low volumes is a consequence of selectively controlling certain properties of the membrane, such as the shape or size of the membrane, the size of the pores, the material from which the membrane is formed (including its surface energy), etc. For example, the membrane 23 may be selected to have any desired shape, such as a generally rectangular, square, circular, or any other regular or irregular shape. In some cases, one shape, such as a rectangular shape, may provide a longer flow time than another shape, such as a circular shape. Specifically, a generally rectangular membrane may have a long length (e.g., dimension that is substantially parallel to the flow of the test sample) and a small width (e.g., dimension that is substantially perpendicular to the flow of the test sample) to impart a slower flow rate. In some embodiments, for example, the width of a generally rectangular membrane may be from about 0.5 to about 10 millimeters, in some embodiments from about 1 to about 5 millimeters, and in some embodiments, from about 1 to about 3 millimeters. The length of such a membrane 23 may be from about 1 to about 40 millimeters, in some embodiments from about 1 to about 20 millimeters, and in some embodiments, from about 1 to about 5 millimeters. The size of the pores may also affect the flow time of a test sample through the membrane 23. Specifically, smaller pore sizes often result in slower flow rates. In most embodiments, the pores of the membrane 23 have an average size of from about 1 micron to about 50 microns, in some embodiments from about 5 microns to about 30 microns, and in some embodiments from about 5 microns to about 15 microns.

The materials used to form the membrane 23 may also affect the flow time of the test sample. Some examples of suitable materials used to form the porous membrane may include, but are not limited to, natural, synthetic, or naturally occurring materials that are synthetically modified, such as polysaccharides (e.g., cellulose materials such as paper and cellulose derivatives, such as cellulose acetate and nitrocellulose); polyether sulfone; polyethylene; nylon; polyvinylidene fluoride (PVDF); polyester; polypropylene; silica; inorganic materials, such as deactivated alumina, diatomaceous earth, MgSO₄, or other inorganic finely divided material uniformly dispersed in a porous polymer matrix, with polymers such as vinyl chloride, vinyl chloride-propylene copolymer, and vinyl chloride-vinyl acetate copolymer; cloth, both naturally occurring (e.g., cotton) and synthetic (e.g., nylon or rayon); porous gels, such as silica gel, agarose, dextran, and gelatin; polymeric films, such as polyacrylamide; and so forth. It should be understood that the term “nitrocellulose” refers to nitric acid esters of cellulose, which may be nitrocellulose alone, or a mixed ester of nitric acid and other acids, such as aliphatic carboxylic acids having from 1 to 7 carbon atoms. Without intending to be limited by theory, it is believed that the rate at which the test sample flows through the membrane 23 may be greater for materials that are more hydrophilic in nature. Thus, for membranes of approximately the same pore size, shape, and dimensions, those made of nitrocellulose may result in a faster flow time than those made of polyvinylidene fluoride, which is somewhat less hydrophilic than nitrocellulose.

To initiate the detection of an analyte, the test sample may be directly applied to the sample pad 21. Alternatively, the test sample may be applied directly to the sample channel 14, or any other component of the assay device 20 that is in fluid communication with the sample channel 14. Although the analyte of interest may be inherently capable of undergoing the desired oxidation/reduction reactions because it contains a redox center, it may be desired, in other embodiments, to attach a redox label to the analyte. The redox label may be applied at various locations of the device 20, such as to an optional conjugate pad 22 so that it may bind to the analyte of interest before reaching the sample channel 14. The conjugate pad 22 is formed from a material through which the test sample is capable of passing. For example, in one embodiment, the conjugate pad 22 is formed from glass fibers. Although only one conjugate pad 22 is shown, it should be understood that additional conjugate pads may also be used in the present invention. Besides the conjugate pad 22, the analyte may be bound to a redox label within the sample channel 14 or any other location of the assay device 20, or even prior to being applied to the device 20.

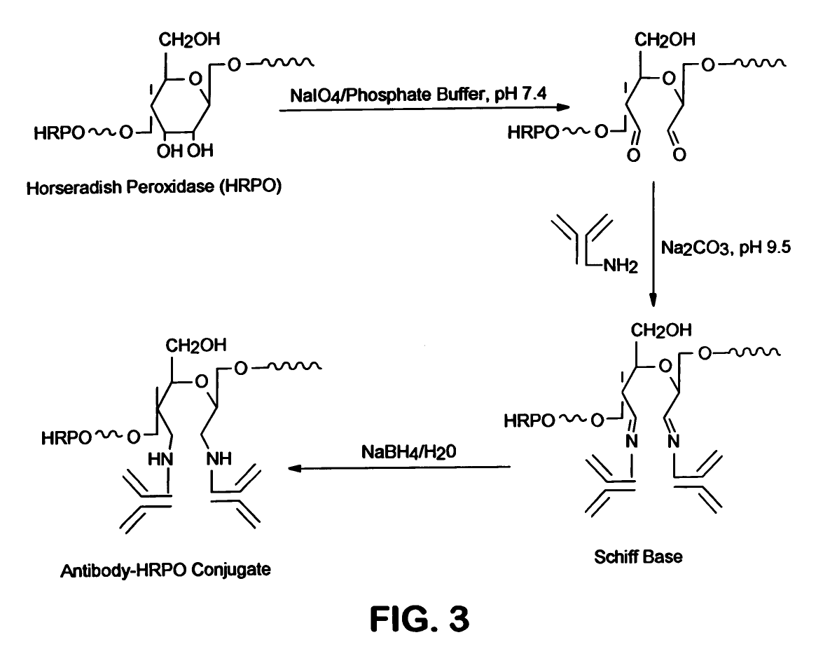

The term “redox label” refers to a compound that has one or more chemical functionalities (i.e., redox centers) that may be oxidized and reduced. Such redox labels are well known in the art and may include, for instance, an enzyme such as alkaline phosphatase (AP), horseradish peroxidase (HRP), glucose oxidase, beta-galactosidase, urease, and so forth. Other organic and inorganic redox compounds are described in U.S. Pat. No. 5,508,171 to Walling, et al.; U.S. Pat. No. 5,534,132 to Vreeke, et al.; U.S. Pat. No. 6,241,863 to Monbouguette; and U.S. Pat. No. 6,281,006 to Heller, et al., which are incorporated herein in their entirety by reference thereto for all purposes. Horseradish peroxidase (HRP), for instance, is an enzyme that is commonly employed in electrochemical affinity assay devices. Two methods are commonly used for the preparation of antibody-coupled horseradish peroxidase (HRP) conjugates, i.e., “glutaraldehyde” and “periodate” oxidation. As is known in the art, the “glutaraladehyde” method involves two steps and results in high molecular weight aggregates. Further, the “periodate” method involves three steps. For instance, as shown in FIG. 3, the “periodate” method may reduce interference of HRP active-site amino groups because it is only conjugated through carbohydrate moieties. Specifically, the “periodate” method opens up the carbohydrate moiety of the HRP glycoprotein molecule to form aldehyde groups that will form Schiff bases with antibody amino groups. Thus, although not required, it may be desired to use HRP formed by the “periodate” method to minimize background current.

Besides being directly attached to the analyte, the redox label may also be indirectly attached to the analyte through a specific binding member for the analyte. Specific binding members generally refer to a member of a specific binding pair, i.e., two different molecules where one of the molecules chemically and/or physically binds to the second molecule. For instance, immunoreactive specific binding members may include antigens, haptens, aptamers, antibodies, and complexes thereof, including those formed by recombinant DNA methods or peptide synthesis. An antibody may be a monoclonal or polyclonal antibody, a recombinant protein or a mixture(s) or fragment(s) thereof, as well as a mixture of an antibody and other specific binding members. The details of the preparation of such antibodies and their suitability for use as specific binding members are well known to those skilled in the art. Other common specific binding pairs include but are not limited to, biotin and avidin, biotin and streptavidin, antibody-binding proteins (such as protein A or G) and antibodies, carbohydrates and lectins, complementary nucleotide sequences (including label and capture nucleic acid sequences used in DNA hybridization assays to detect a target nucleic acid sequence), complementary peptide sequences including those formed by recombinant methods, effector and receptor molecules, hormone and hormone binding protein, enzyme cofactors and enzymes, enzyme inhibitors and enzymes, and so forth. Furthermore, specific binding pairs may include members that are analogs of the original specific binding member. For example, a derivative or fragment of the analyte, i.e., an analyte-analog, may be used so long as it has at least one epitope in common with the analyte.

The redox labels may be used in a variety of ways to form a probe. For example, the redox labels may be used alone to form probes. Alternatively, the redox labels may be used in conjunction with polymers, liposomes, dendrimers, and other micro- or nano-scale structures to form probes. For example, the redox labels may be used in conjunction with particles (sometimes referred to as “beads”) to form the probes. Naturally occurring particles, such as nuclei, mycoplasma, plasmids, plastids, mammalian cells (e.g., erythrocyte ghosts), unicellular microorganisms (e.g., bacteria), polysaccharides (e.g., agarose), and so forth, may be used. Further, synthetic particles may also be utilized. For example, in one embodiment, latex particles are utilized. Although any latex particle may be used in the present invention, the latex particles are typically formed from polystyrene, butadiene styrenes, styreneacrylic-vinyl terpolymer, polymethylmethacrylate, polyethylmethacrylate, styrene-maleic anhydride copolymer, polyvinyl acetate, polyvinylpyridine, polydivinylbenzene, polybutyleneterephthalate, acrylonitrile, vinylchloride-acrylates, and so forth, or an aldehyde, carboxyl, amino, hydroxyl, or hydrazide derivative thereof. Other suitable particles may be described in U.S. Pat. No. 5,670,381 to Jou, et al. and U.S. Pat. No. 5,252,459 to Tarcha, et al., which are incorporated herein in their entirety by reference thereto for all purposes. In addition, inorganic particles, such as colloidal metallic particles (e.g., gold) and non-metallic particles, carbon particles, and the like, may also be utilized. The mean diameter of the particles may generally vary as desired. For example, in some embodiments, the mean diameter of the particles may range from about 0.01 microns to about 1,000 microns, in some embodiments from about 0.01 microns to about 100 microns, and in some embodiments, from about 0.01 microns to about 10 microns. In one particular embodiment, the particles have a mean diameter of from about 0.01 to about 2 microns. Generally, the particles are substantially spherical in shape, although other shapes including, but not limited to, plates, rods, bars, irregular shapes, etc., are suitable for use in the present invention. As will be appreciated by those skilled in the art, the composition, shape, size, and/or density of the particles may widely vary.

Once labeled, if desired, the analyte of interest may then travel through the sample channel 14 until it reaches a detection zone 31 that is defined by the substrate 40. Various electrodes are formed on the substrate 40 within the detection zone 31. Specifically, as shown, a detection working electrode 42, a counter electrode 46, a reference electrode 48, and an optional calibration working electrode 44 are formed on the substrate 40. Leads 43 for the electrodes are disposed parallel to the flow of the test sample. Alternatively, the leads 43 may be positioned perpendicular to the flow of the test sample. If desired, the reference and counter electrodes 46 and 48 may be combined into a single “pseudo” electrode. This may be particularly beneficial when the solution resistance is negligible or the generated current is relatively small. Moreover, it should be understood that each working electrode 42 and 44 may be paired with a separate counter and reference electrode. Further, multiple detection and calibration working electrodes 42 and 44 may be utilized.

The detection working electrode 42 is typically formed from a thin film of conductive material disposed on the substrate 40. Generally speaking, a variety of conductive materials may be used to form the detection working electrode 42. Suitable materials include, for example, carbon, metals (e.g., platinum, palladium, gold, tungsten, titanium, etc.), metal-based compounds (e.g., oxides, chlorides, etc.), metal alloys, conductive polymers, combinations thereof, and so forth. Particular examples of carbon electrodes include glassy carbon, graphite, mesoporous carbon, nanocarbon tubes, fullerenes, etc. Thin films of these materials may be formed by a variety of methods including, for example, sputtering, reactive sputtering, physical vapor deposition, plasma deposition, chemical vapor deposition (CVD), printing, spraying, and other coating methods. For instance, carbon or metal paste based conductive materials are typically formed using screen printing, which either may be done manually or automatically. Likewise, metal-based electrodes are typically formed using standard sputtering or CVD techniques, or by electrochemical plating.

Discrete conductive elements may be deposited to form the detection working electrode 42, for example, using a patterned mask. Alternatively, a continuous conductive film may be applied to the substrate and then the detection working electrode 42 may be patterned from the film. Patterning techniques for thin films of metal and other materials are well known in the art and include photolithographic techniques. An exemplary technique includes depositing the thin film of conductive material and then depositing a layer of a photoresist over the thin film. Typical photoresists are chemicals, such as organic compounds, that are altered by exposure to light of a particular wavelength or range of wavelengths. Exposure to light makes the photoresist either more or less susceptible to removal by chemical agents. After the layer of photoresist is applied, it is exposed to light, or other electromagnetic radiation, through a mask. Alternatively, the photoresist is patterned under a beam of charged particles, such as electrons. The mask may be a positive or negative mask depending on the nature of the photoresist. The mask includes the desired pattern of working electrodes, which are the electrodes on which the electrocatalytic reactions take place when the detection marker and the redox label are both present and immobilized on the electrode. Once exposed, the portions of the photoresist and the thin film between the working electrode 42 is selectively removed using, for example, standard etching techniques (dry or wet), to leave the isolated working electrode of the array.

The detection working electrode 42 may have a variety of shapes, including, for example, square, rectangular, circular, ovoid, and so forth. The detection working electrode 42 may have varying dimensions (e.g., length, width, or diameter). In some embodiments, the width of the electrode 42 may be from about 0.5 to about 10 millimeters, in some embodiments from about 1 to about 5 millimeters, and in some embodiments, from about 1 to about 3 millimeters. If desired, one or more dimensions of the electrode 42 may be selected to correspond to a dimension of the membrane 23. In this manner, most if not all of the test sample flowing through the membrane 23 will contact a surface of the electrode 42, which alleviates possible background interference that might otherwise result due to the test sample flowing around the edges of the electrode 42. Alternatively, the electrode 42 and the membrane 23 may have different “actual” widths, but have substantially the same “effective” widths in that the portion of their widths exposed to the flow of the test sample is substantially the same. For instance, the width of the membrane 23 may actually be larger than the width of the electrode 42. Nevertheless, the portion of the membrane's width that is larger than that of the electrode 42 may be blocked to the flow of the test sample using, for instance, tape.

The surface smoothness and layer thickness of the electrode 42 may be controlled through a combination of a variety of parameters, such as mesh size, mesh angle, and emulsion thickness when using a printing screen. Emulsion thickness may be varied to adjust wet print thickness. The dried thickness may be slightly less than the wet thickness because of the vaporization of solvents. In some embodiments, for instance, the dried thickness of the printed electrode 42 is less than about 100 microns, in some embodiments less than about 50 microns, in some embodiments less than about 20 microns, in some embodiments less than about 10 microns, and in some embodiments, less than about 1 micron.

In addition, one or more surfaces of the detection working electrode 42 are generally treated with various affinity reagents. For example, in one embodiment, the surface of the detection working electrode 42 is treated with a specific binding capture ligand. The specific binding capture ligand is capable of directly or indirectly binding to the analyte of interest. The specific binding capture ligand typically has a specificity for the analyte of interest at concentrations as low as about 10⁻⁷ moles of the analyte per liter of test sample (moles/liter), in some embodiments as low as about 10⁻⁸ moles/liter, and in some embodiments, as low as about 10⁻⁹ moles/liter. For instance, some suitable immunoreactive specific binding capture ligands may include antigens, haptens, aptamers, antibodies, and complexes thereof, including those formed by recombinant DNA methods or peptide synthesis. Generally speaking, electrochemical stability is desired for accurate analyte detection because any redox response from the specific binding capture ligand may complicate the true current responses from the analyte. Thus, in most embodiments, the specific binding capture ligand is stable at the potential range of from −0.75 to +0.75 Volts, in some embodiments from −0.50 to +0.50 Volts, and in some embodiments, from −0.35 to +0.35 Volts, in comparison with the reference electrode.

Besides specific binding capture ligands, redox mediators may also be applied to the surface of the detection working electrode 42. The redox mediators may be applied to the working electrode 42 at any time, such as during formation of the assay device or during testing. In one embodiment, for instance, the redox mediator is immobilized on the surface of the electrode 42. Alternatively, the redox mediator is applied to the surface only after the test sample reaches the detection zone 31. Some examples of suitable redox mediators that may be used in the present invention include, but are not limited to, oxygen, ferrocene derivatives, quinones, ascorbic acids, redox polymers with metal complexes, glucose, redox hydrogel polymers, and organometallic complexes based upon osmium, ruthenium, iron, etc., and so forth. Particular examples of suitable redox mediators include ferricyanide, 2,5-dichloro-1,4-benzoquinone, 2,6-dichloro-1,4-benzoquinone, 2,6-dimethyl-1,4-benzoquinone, phenazine ethosulfate, phenazine methosulfate, phenylenediamine, 1-methoxy-phenazine methosulfate, and 3,3′5,5′ tetramethyl benzidine (TMB). Substrates may also be used in conjunction with a soluble redox mediator present in solution. In such instances, the solution-based substrate may be simply placed on the surface of the applicable electrode. Some commercially available examples of such solution-based substrates include 1-Step turbo TMB (Pierce Chemical Co., Rockford, Ill.) and K-Blue Substrate Ready-to-Use (Neogen Corp., Lexington, Ky.). For instance, “K-Blue Substrate” is a chromogenic substrate for horseradish peroxidase that contains 3,3′,5,5′ tetramethylbenzidine (TMB) and hydrogen peroxide (H₂O₂). Other suitable redox mediators are described in U.S. Pat. No. 6,281,006 to Heller, et al.; U.S. Pat. No 5,508,171 to Walling, et al.; U.S. Pat. No. 6,080,391 to Tsuchiya, et al.; and U.S. Pat. No 6,461,496 to Feldman, et al., which are incorporated herein in their entirety by reference thereto for all purposes. As will be readily recognized by those skilled in the art, many other different reaction mechanisms may be used in the present invention to achieve the electrolysis of an analyte through a reaction pathway incorporating a redox mediator.

The affinity reagents may be applied to the surface of the detection working electrode 42 using a variety of well-known techniques. For example, the reagents may be directly immobilized on the surface of the electrode 42, may be contained within a substrate that is disposed on the surface of the electrode 42, may be mixed into the materials used to form the electrode 42, and so forth. In one embodiment, the affinity reagents are formulated into a solution and screen-printed, ink-jet printed, drop coated, or sprayed onto the working electrode surface. Screen printing inks, for instance, are typically formulated in a buffer solution (e.g., phosphate buffer) containing the specific or non-specific binding members. Although not required, an organic immobilizing solvent may be added to the aqueous buffer solution to help wet the hydrophobic or non-hydrophilic surfaces. In some embodiments, for example, the solvent may be an alcohol, ether, ester, ketone, or combinations thereof. When coated, the electrode 42 is desirably applied with a uniform coating across its entire surface. The coating is typically a single layer, but multiple layers are also contemplated by the present invention. The coating, regardless of monolayer or multiple layers, is typically optimized to give the largest current and signal/noise ratio.

Upon application to the electrode surface, the reagents may optionally be stabilized. Stabilization facilitates long-term stability, particularly for ensuring required shelf-life incurred during shipping and commercial selling. For instance, in one embodiment, stabilization may be accomplished by coating a layer, such as a polymer, gel, carbohydrate, protein, etc., onto the electrode surface before and/or after application of the affinity reagent(s). Some commercially available examples of such a stabilization coating are Stabilcoat®, Stabilguard®, and Stabilzyme® from Surmodics, Inc. of Eden Prairie, Minnesota.

Besides a detection working electrode 42, the substrate 40 may optionally include a calibration working electrode 44. When utilized, the calibration working electrode 44 may enhance the accuracy of the analyte concentration determination. For instance, a current will generally be generated at the calibration working electrode 44 that corresponds to intrinsic background interference stemming from the counter and reference electrodes, as well as the working electrodes themselves. Once determined, the value of this intrinsic background current may be used to calibrate the measured current value at the detection working electrode 42 to obtain a more accurate reading. The calibration working electrode 44 may generally be formed as described above with respect to the detection working electrode 42. In fact, because the calibration working electrode 44 is configured to calibrate the detection working electrode 42, it is generally desired that such electrodes are formed in approximately the same manner, from the same materials, and to have the same shape and/or size.

The detection and calibration working electrodes 42 and 44 are also generally applied with the same surface treatments to improve the calibration accuracy. However, one primary difference between the detection working electrode 42 and the calibration working electrode 44 is that the electrode 44 does not typically contain a specific binding capture ligand for the analyte of interest. This allows most, if not all, of the analyte to bind to the electrode 42, thereby enabling the electrode 42 to be used primarily for detection and the electrode 44 to be used primarily for calibration.

For example, the use of this calibration electrode 44 would help determine if non-specific binding was occurring on the electrode surfaces. In some instances, non-specific binding of the redox label or other current-generating compounds to the capture ligand present on the detection working electrode 42 may create inaccuracies in the measured current. Contrary to the specific binding ligands, the non-specific binding ligands do not have a high specificity for the analyte of interest. In fact, the non-specific binding capture ligand typically has no specificity for the analyte of interest at concentrations as high as about 10−2 moles of the analyte per liter of test sample (moles/liter), and in some embodiments, as high as about 10−3 moles/liter. The non-specific binding ligands may form bonds with various immunoreactive compounds. These immunoreactive compounds may have a redox center or may have inadvertently been provided with a redox center through attachment of a redox compound (e.g., enzyme). Without the calibration working electrode 44, these immunoreactive compounds would thus generate a low level of current detected from the detection working electrode 42, which causes error in the resulting analyte concentration calculated from the generated current. This error may be substantial, particularly when the test sample contains a low analyte concentration.

To minimize any undesired binding (including non-specific binding as described above) on the surfaces of the working electrodes 42 and 44, the counter electrode 46, or the reference electrode 48, a blocking agent may be applied thereto. The term “blocking agent” means a reagent that adheres to the electrode surface so that it “blocks” or prevents certain materials from binding to the surface. Blocking agents may include, but are not limited to, β-casein, Hammerstern-grade casein, albumins such as bovine serum albumin, gelatin, pluronic or other surfactants, polyethylene glycol, polyvinyl pyrrolidone or sulfur derivatives of the above compounds, a surfactant such as Tween 20, 30, 40 or Triton X-100, a polymer such as polyvinyl alcohol, and any other blocking material known to those of ordinary skill in the art. This includes commercial blends, such as SuperBlock® or SEA BLOCK (Pierce Chemical Co., Rockford, Ill.) or Heterophilic Blocking Reagent (Scantibodies, Santee, Calif.). Depending on the conductive materials used for preparing the working electrodes, the blocking agents may be formulated to adapt to the electrode surface properties. In some embodiments, a cocktail containing multiple blocking agents may be applied onto an electrode and incubated for 5 to 30 minutes, and any excess solution may be removed and the resulting electrode thoroughly dried.

In general, a variety of assay formats may be used in the present invention. In this regard, various embodiments of the present invention will now be described in more detail. It should be understood, however, that the embodiments discussed below are only exemplary, and that other embodiments are also contemplated by the present invention. For instance, referring again to FIG. 1, the test sample is initially applied to the sample pad 21 and travels to the conjugate pad 22. At the conjugate pad 22, any analyte within the test sample mixes with and attaches to a redox label. In one embodiment, for instance, the label is horseradish peroxidase (HRP) and the analyte of interest is glucose. Because the conjugate pad 22 is in fluid communication with the sample channel 14, the labeled analyte may migrate from the conjugate pad 22 to the sample channel 14, and then to the detection zone 31 present on the substrate 40. The labeled analyte binds to the specific binding capture ligand on the detection working electrode 42 where it reacts with a redox mediator. In one embodiment, for example, the analyte is reacted as follows:
Analyte (reduced form)+Redox Mediator (oxidized form)→Analyte (oxidized form)+Redox Mediator (reduced form)

In addition, non-specific binding may be monitored and corrected using the optional calibration working electrode 44. It is intended that the amount of non-analyte materials that bind to the calibration working electrode 44 will be similar to the amount of non-analyte material that non-specifically binds to the detection working electrode 42. Thus, in this manner, the background signal due to non-specific binding may be compensated. In one embodiment, for example, the non-analyte biological materials (abbreviated “NAB”) are reacted as follows:
NAB (reduced form)+Redox Mediator (oxidized form)→NAB (oxidized form)+Redox Mediator (reduced form)

Detection techniques, such as amperometric, couloumetric, voltammetric, etc., may then be used to detect the analyte. A further description of such electrochemical detection techniques is described in Electrochemical Methods, A. J. Bard and L. R. Faulner, John Wiley & Sons (1980). In one embodiment, for example, a potentiostat or reader may apply a potential difference between the detection working electrode 42 and counter electrode 46. When the potential difference is applied, the amount of the oxidized form of the redox mediator at the counter electrode 46 and the potential difference is sufficient to cause diffusion limited electro-oxidation of the reduced form of the redox mediator at the surface of the detection working electrode 42. The magnitude of the required potential is thus dependent on the redox mediator. Namely, the potential is typically large enough to drive the electrochemical reaction to or near completion, but not large enough to induce significant electrochemical reaction of interferents, such as urate, ascorbate, and acetaminophen, that may affect the current measurements. Similarly, the potential difference may also be supplied between the optional calibration working electrode 44 and counter electrode 46. When the potential difference is applied, diffusion limited electro-oxidation of the reduced form of the redox mediator occurs at the surface of the calibration working electrode 44.

Generally, the detection and calibration working electrodes 42 and 44 simultaneously generate a respective signal from a single measurement of a sample. The simultaneously generated signals are averaged by a processing circuit, such as a multi-channel potentiostat. Multi-channel potentiostats are well known in the art, and are described, for instance, in U.S. Pat. No. 5,672,256 to Yee, which is incorporated herein in its entirety by reference thereto for all purposes. Each channel of a multi-channel potentiostat may function as a potentiostat, and thus may be associated with its own reference and/or counter electrode, or may share reference and/or counter electrodes. One suitable example of a multi-channel potentiostat that may be used in the present invention is commercially available under the name “MSTAT” from Arbin Instruments, Inc. of College Station, Texas. Once detected, the current measured at the detection working electrode 42 is calibrated by the current measured at the calibration working electrode 44 to obtain a calibrated current reading that may be correlated to the concentration of analyte in the sample. The correlation may result from predetermined empirical data or an algorithm, as is well known in the art. If desired, the generated current and analyte concentration may be plotted as a curve to aid in the correlation therebetween. As a result, calibration and sample testing may be conducted under approximately the same conditions at the same time, thus providing reliable quantitative or semi-quantitative results, with increased sensitivity. In the case of a sandwich assay format, the signal provided by the detection working electrode 42 is directly proportional to the analyte concentration in the test sample. In the case of a competitive assay format, which may, for instance, be constructed by applying a labeled analyte on the surface of the detection working electrode 42, the signal provided by the detection working electrode 42 is inversely proportional to the analyte concentration in the test sample. It should be understood that the potential may be applied either before or after the sample has been placed in the detection area (e.g. electrodes). The potential is preferably applied after the sample has reached the detection area to prevent continued electrochemical process during the formation of immunocomplex on the electrode surface. The formation time may be from about 1 second to about 15 minutes, depending on the sample size, channel, size, membrane size, and/or electrode size.

Various parameters of the detection technique may be utilized to improve the consistency and accuracy of the assay device. For example, variations of fabrication processes, such as electrode coating, flow control, sample size, mediator efficiency, etc, may have an impact on data collection. Thus, in one embodiment, the time at which current readings are measured may be selected to achieve improved results. Specifically, when a potential is applied, the initial reading of the current may be inaccurate or less reliable. Accordingly, the time at which the current reading is first recorded may be after applying the potential. Thus, in some embodiments, the first recording is from about 0.001 seconds to about 10 minutes, in some embodiments from about 0.1 seconds to about 1 minute, in some embodiments from about 0.5 to about 20 seconds, and in some embodiments, from about 1 to about 10 seconds, after applying the potential. In addition, the current readings may also be recorded in flexible time intervals. If desired, for example, the number of readings taken at the beginning of the recordings may be greater than the number taken at the end. This is due primarily to the fact that, at the later stages of the recordings, the decrease in measured current is usually more profound than the magnitude of the potential pulse.

Regardless of the detection environment, the total charge is normally the same for a given analyte concentration because the current measurements are obtained at intervals over the course of the entire assay and integrated over time to obtain the total amount of charge, Q, passed to or from the electrode. Q is then used to calculate the concentration of the analyte. For instance, the total charge, Q, may be directly calculated when the redox label is able to generate a detection signal. The completion of the electrochemical reaction is signaled when the current reaches a steady-state value that indicates all or nearly all of the redox labels on the electrode surface have been electrolyzed. In such cases, at least 90%, in some embodiments at least 95%, and in some embodiments, at least 99% of the complexes are electrolyzed. In other cases, however, the redox label may not be able to generate a measurable detection signal without amplification. For instance, an enzyme label may require a substrate to provide amplification of the detection current. If desired, the substrate may be used in excess to ensure that the detection signal reaches the a measurable level. In some embodiments, for example, the ratio of the substrate to the complexes formed on the electrode surface is at least 10:1, in some embodiments at least 100:1, in some embodiments at least 1,000:1, and in some embodiments, at least 10,000:1.

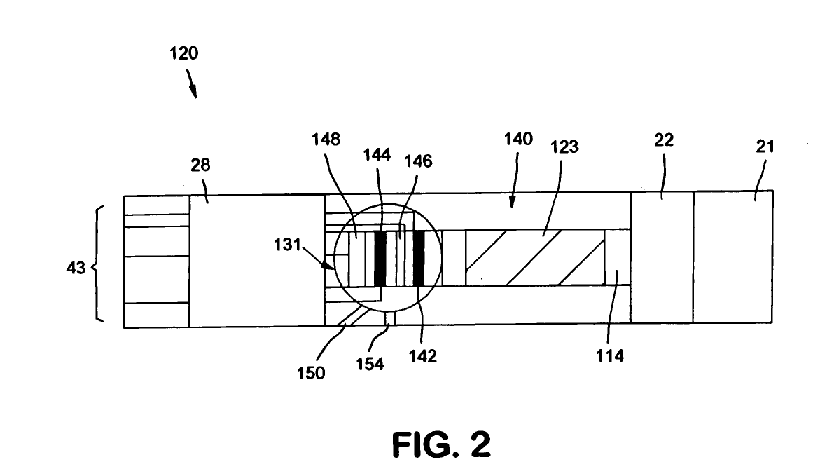

Referring to FIG. 2, another embodiment of an assay that may be performed according to the present invention is illustrated. In this embodiment, a lateral flow assay device 120 is shown that includes a substrate 140 on which is formed a sample channel 114 and a porous membrane 123. As described above, the sample channel 114 and porous membrane 123 facilitate the flow of the test sample to a detection zone 131. The assay device 120 also includes other channels that serve a variety of purposes. For example, the assay device 120 includes a washing channel 150 that provides for the flow of a washing reagent to the detection zone 131 to remove any redox labels that remain unbound at the detection zone 131. Examples of washing agents may include, for instance, water, a buffer solution, such as PBS buffer, HEPES buffer, etc., and so forth. In addition, a reagent channel 154 may also be provided through which affinity reagents (e.g., capture ligands, redox mediators, particles, labels, etc.) may flow to initiate a desired electrochemical reaction. If desired, the additional washing channel 150 and reagent channel 154 may be printed in the manner described above.

By using separate and distinct sample addition, washing, and reagent channels, controlled and sequential delivery of different solutions may be provided. For example, in one embodiment, a test sample is first supplied to the sample channel 114, where an analyte within the test sample is allowed to mix with and bind to a redox label. The test sample then flows to the detection zone 131 so that the labeled analyte binds to a specific binding ligand immobilized on a detection working electrode 142. Thereafter, a washing reagent is supplied to the washing channel 150 to remove any free redox labels that do not bind to the detection working electrode 142. This inhibits the assay device 120 from giving a false positive signal. After washing, one or more affinity reagents are supplied to the reagent channel 154 to initiate the electrochemical reaction within the detection zone 131.

As an alternative to sequentially applying reagents in the manner described above, simultaneous application may also be employed. In such instances, the time at which the different reagents arrive at the detection zone 131 is selectively controlled using other mechanisms. For example, membranes of different pore sizes, channels of different sizes, surface energies, etc., may be used to control when each particular reagent reaches the detection working electrode 142.

Although various embodiments of assay formats and devices have been described above, it should be understood, that the present invention may utilize any assay format or device desired, and need not contain all of the components described above. Further, other well-known components of assay formats or devices not specifically referred to herein may also be utilized in the present invention. For example, various assay formats and/or devices are described in U.S. Pat. No. 5,508,171 to Walling, et al.; U.S. Pat. No. 5,534,132 to Vreeke, et al.; U.S. Pat. No. 6,241,863 to Monbouquette; U.S. Pat. No. 6,270,637 to Crismore, et al.; U.S. Pat. No. 6,281,006 to Heller, et al.; and U.S. Pat. No. 6,461,496 to Feldman, et al., which are incorporated herein in their entirety by reference thereto for all purposes.

In addition, it should be understood that both sandwich and competitive assay formats may be formed according to the present invention. Techniques and configurations of sandwich and competitive assay formats are well known to those skilled in the art. For instance, sandwich assay formats typically involve mixing the test sample with labeled antibodies so that complexes of the analyte and the labeled antibody are formed. These labeled complexes contact a detection zone where they bind to another antibody and become immobilized, thereby indicating the presence of the analyte. Some examples of such sandwich-type assays are described by U.S. Pat. Nos. 4,168,146 to Grubb, et al. and U.S. Pat. No. 4,366,241 to Tom, et al., which are incorporated herein in their entirety by reference thereto for all purposes. In a competitive assay, a labeled analyte or analyte-analog competes with an unlabeled analyte in the test sample for binding to a ligand immobilized at the detection zone. Competitive assays are typically used for detection of analytes such as haptens, each hapten being monovalent and capable of binding only one antibody molecule. Examples of competitive immunoassay devices are described in U.S. Pat. No. 4,235,601 to Deutsch, et al., U.S. Pat. No. 4,442,204 to Liotta, and U.S. Pat. No. 5,208,535 to Buechler, et al., which are incorporated herein in their entirety by reference thereto for all purposes.

The present invention provides a low-cost, flow-through assay device that may provide accurate analyte detection. The assay devices of the present invention may be produced as a single test for detecting an analyte or it may be formatted as a multiple test device. The uses for the assay devices of the present invention include, but are not limited to, detection of chemical or biological contamination in garments, such as diapers, the detection of contamination by microorganisms in prepacked foods such as fruit juices or other beverages, and the use of the assay devices of the present invention in health diagnostic applications such as diagnostic kits for the detection of antigens, microorganisms, and blood constituents. It should be appreciated that the present invention is not limited to any particular use or application.

The present invention may be better understood with reference to the following examples.

### EXAMPLE 1

Electrodes were printed onto Mylar® substrates obtained from DuPont. The substrates had a length of 0.5 centimeters and a width of 1.5 centimeters. Carbon (7101 or 7102), silver (5000), and silver/silver chloride (5847) inks were obtained from DuPont Biosensor Group of Research Triangle Park, North Carolina. For printing the inks, a screen frame was first fixed onto a screen frame holder and adjusted. Initially, a silver ink line was printed on the substrate to enhance the conductivity between the printed leads and electrodes. Thereafter, carbon ink was printed over the silver ink line to form a detection working electrode, calibration working electrode, and counter electrode. The silver/silver chloride ink was printed onto the substrate to form a reference electrode. Leads for the electrodes were also printed. The resulting electrode strips were left at room temperature for 2 hours, and then heated at 37° C. for 2 hours. The temperature was then raised to 60° C. and dried an additional 2 hours. Thereafter, the temperature was again raised to between 120 to 140° C. for 20 minutes. Such stepwise drying helped achieve high uniformity of the electrode surface, while also removing residue solvents of the original ink formulations. The dried electrode strips were then kept either in a plastic bag or in a desiccator.

### EXAMPLE 2

The ability to print flow channels onto the electrode strip of Example 1 was demonstrated. Specifically, an insulation layer and flow channel were then printed simultaneously onto the substrates using a UV-curable dielectric composition available from DuPont under the name “5018G.” Printing was performed with a screen printer available from Affiliated Manufacturers, Inc. (“AMI-Presco”) of North Branch, N.J. under the name “HC-53.” The screen frame utilized had a size of 5×7 inches, a mesh size of 80×0.0037 to 400×0.0007, and a stencil angle of 22 to 45 degrees. The insulation layer essentially covered the substrate area not otherwise covered by the electrodes, leads, or flow channels. The resulting flow channel had a length of 2.5 centimeters and a width of 0.5 centimeters. The height of the flow channel ranged from about 10 to about 150 micrometers, and was measured using a micrometer available from Mitutoyo America Corporation of Aurora, Ill. The insulation layer and channel were then cured by placing the substrate under a solar simulator, which is available from Solar Light Co. of Philadelphia, Pa. under the name “LS 1000-4R-UV”, for about 5 to 10 seconds.

### EXAMPLE 3

Membrane strips of a nylon mesh membrane (30 mesh size, commercially available from Millipore Corp. of Billerica, Mass.) were provided that had a width of 4.5 centimeters and a length of 15 centimeters. To the bottoms of the strips, two glass fiber pads (sample and conjugate pads) were attached using tape. The conjugate pad was in direct contact with the membrane, and the sample pad was in direct contact with the conjugate pad. The conjugate pad was treated with 3 microliters of LH-α-HRP monoclonal antibody conjugate (5 micrograms per milliliter in PBS buffer) and dried for 30 minutes. The LH-α-HRP monoclonal antibody conjugate was obtained from Fitzgerald Industries Int'l of Concord, Mass. The membrane strips were placed onto a sampling instrument commercially available from Kinematic Automation of Twain Harte, Calif. under the name “Matrix 2210 (Universal Laminator).” Thereafter, the strips were cut into individual strips having a width ranging from 1 to 10 millimeters using a strip cutter commercially available from Kinematic Automation under the name “Matrix 2360.”

### EXAMPLE 4

The ability to control the flow of a test sample in accordance with the present invention was demonstrated. Initially, an electrode strip was formed in accordance with Example 1. 0.5 microliters of LH-α-HRP monoclonal antibody conjugate (Fitzgerald Industries Int′ l of Concord, Mass.) was then drop coated onto the surface of the detection working electrode with an Eppendorf microliter pipette. The LH-α-HRP monoclonal antibody conjugate had a concentration of about 5 nanograms per milliliter in a mixture of 80% PBS buffer and 20% isopropanol, and had a pH of 7.4. The resulting electrode strip was then placed at room temperature and allowed to air dry. Thereafter, the coated detection working electrode was treated with 3 microliters of a protein stabilizing formulation (20 wt. % Stabilcoat™ from SurModics, Inc. of Eden Prairie, Minn. and 0.05 wt. % Tween 20 in a PBS buffer, pH of 7.4). The incubation time was 15 minutes. After incubation, the remaining solution was removed by a wicking material, and the electrode strip was dried under an air stream. In addition, the entire detection area, including the working, calibration, counter, and reference electrodes, was treated with about 100 microliters of a solution containing β-casein (1 wt. %), Tween 20 (0.05 wt. %), and PBS buffer (pH of 7.4), and dried.

Once formed, the treated electrode strip was attached to a membrane strip of Example 3. Specifically, the membrane strip was placed over the flow channel. A wicking pad was also attached downstream from the electrodes having a length of 0.5 centimeters and a width of 1.5 centimeters. The strips and wicking pad were attached using a covering tape that allowed the test sample to flow to the electrodes through a path defined by the membrane. The covering tape had a length of 3 centimeters and a width of 1.5 centimeters, and is commercially available from Adhesives Research, Inc. of Glen Rock, Pa. under the name “ARcare®.”

Thereafter, a test sample was applied to the sample pads in an amount of 100 microliters. The test sample contained an LH antigen in a concentration of 100 nanograms per milliliter in PBS buffer (pH of 7.42). The assay was allowed to develop until the wicking pad had absorbed almost all of the fluid from the test sample, which occurred in about 2 to about 15 minutes. A TMB substrate solution was then applied to the working electrodes in an amount of 30 microliters. Thereafter, a potential of about 0.1 to 0.3 volts was applied using a multi-channel VMP potentiostat commercially available from Perkin-Elmer, Inc. of Wellesley, Mass. The current was recorded after about 20 seconds, and effectively indicated the presence of the LH antigen.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

1-49. (canceled)

50. A method for detecting the presence or quantity of an analyte residing in a test sample, said method comprising:
i) forming a flow-through assay device by a method comprising:
a) printing a fluidic channel onto a surface of a substrate, wherein said channel has opposing walls that are raised above the surface of the substrate;
b) forming a detection working electrode on said surface of said substrate, said detection working electrode being in fluid communication with said fluidic channel; and
c) treating a surface of said detection working electrode with a specific binding capture ligand for the analyte; and
ii) contacting the test sample with said fluidic channel of said assay device.

51. The method of claim 50, further comprising applying a potential difference between said detection working electrode and a counter electrode to generate a detection current.

52. The method of claim 51, further comprising measuring the detection current generated at said detection working electrode.

53. A method of forming a flow-through assay device for detecting the presence or quantity of an analyte residing in a test sample, said method comprising:
i) printing a fluidic channel onto a surface of a substrate, wherein said channel has opposing walls that are raised above the surface of the substrate;
ii) forming a detection working electrode on said surface of said substrate, said detection working electrode being in fluid communication with said fluidic channel; and
iii) treating a surface of said detection working electrode with a specific binding capture ligand for the analyte.

54. The method of claim 53, wherein said channel is formed from a dielectric material.

55. The method of claim 54, wherein said channel is formed from a polymer.

56. The method of claim 53, wherein the characteristic dimension of said channel is from about 0.5 to about 500 micrometers.

57. The method of claim 53, wherein the characteristic dimension of said channel is from about 1 to about 200 micrometers.

58. The method of claim 53, wherein the characteristic dimension of said channel is from about 5 to about 100 micrometers.

59. The method of claim 53, wherein said channel has a height ranging from about 0.1 to about 500 micrometers.

60. The method of claim 53, wherein said channel has a height of from about 0.5 to about 250 micrometers.

61. The method of claim 53, wherein said channel has a height of from about 1 to about 100 micrometers.

62. The method of claim 53, wherein said channel has a length of from about 1 millimeter to about 50 centimeters.

63. The method of claim 53, wherein said channel has a length of from about 5 millimeters to about 50 millimeters.

64. The method of claim 53, wherein said channel is printed onto said substrate using a contact printing technique.

65. The method of claim 63, wherein said channel is printed onto said substrate using stamp printing or screen printing.

66. The method of claim 53, wherein said channel is printed onto said substrate using a non-contact printing technique.

67. The method of claim 66, wherein said channel is printed onto said substrate using ink-jet printing.

68. The method of claim 53, wherein a porous membrane is placed in fluid communication with said fluidic channel to assist with the flow of the test sample therethrough.

69. The method of claim 53, wherein a redox label is incorporated into the assay device for directly or indirectly binding to the analyte.

70. The method of claim 69, wherein said redox label is an enzyme selected from the group consisting of alkaline phosphatase, horseradish peroxidase, glucose oxidase, beta-galactosidase, urease, and combinations thereof.

71. The method of claim 69, wherein said redox label is used in conjunction with a particle modified with a specific binding member for the analyte.

72. The method of claim 53, wherein said specific binding capture ligand is selected from the group consisting of antigens, haptens, aptamers, antibodies, and complexes thereof.

73. The method of claim 53, wherein said surface of said detection working electrode is treated with a redox mediator.

74. The method of claim 73, wherein said redox mediator is selected from the group consisting of oxygen, ferrocene derivatives, quinones, ascorbic acids, redox polymers with metal complexes, glucose, redox hydrogel polymers, and organometallic complexes.

75. The method of claim 53, further comprising forming at least one additional fluidic channel on said surface of said substrate.

76. A method of forming a flow-through assay device for detecting the presence or quantity of an analyte residing in a test sample, said method comprising:
i) printing a fluidic microchannel onto a surface of a substrate, said fluidic microchannel being formed from a dielectric material and having opposing walls that are raised above the surface of the substrate, wherein said microchannel has a characteristic dimension of from about 0.5 to about 500 micrometers;
ii) forming a detection working electrode on said surface of said substrate, said detection working electrode being in fluid communication with said fluidic microchannel; and
iii) treating a surface of said detection working electrode with a specific binding capture ligand for the analyte.

77. The method of claim 76, wherein said microchannel is formed from a polymer.

78. The method of claim 76, wherein the characteristic dimension of said microchannel is from about 1 to about 200 micrometers.

79. The method of claim 76, wherein the characteristic dimension of said microchannel is from about 5 to about 100 micrometers.

80. The method of claim 76, wherein said microchannel has a height of from about 0.1 to about 500 micrometers.

81. The method of claim 76, wherein said microchannel has a height of from about 1 to about 100 micrometers.

82. The method of claim 76, wherein said microchannel has a length of from about 5 millimeters to about 50 millimeters.

83. The method of claim 76, wherein said microchannel is printed onto said substrate using a contact printing technique.

84. The method of claim 76, wherein said microchannel is printed onto said substrate using a non-contact printing technique.

85. The method of claim 76, wherein a porous membrane is placed in fluid communication with said fluidic microchannel to assist with the flow of the test sample therethrough.

86. The method of claim 76, wherein a redox label is incorporated into the assay device for directly or indirectly binding to the analyte.

87. The method of claim 76, wherein said surface of said detection working electrode is treated with a redox mediator.

88. The method of claim 76, further comprising printing at least one additional fluidic channel onto said surface of said substrate.

89. A method for detecting the presence or quantity of an analyte residing in a test sample, said method comprising contacting the test sample with the fluidic microchannel of claim 76.

90. The method of claim 89, further comprising applying a potential difference between said detection working electrode and a counter electrode to generate a detection current.

91. The method of claim 90, further comprising measuring the detection current generated at said detection working electrode.
